(19) United States
(12) Patent Application Publication
(10) Pub. No.: US 2002/0134393 A1
(43) Pub. Date: Sep. 26, 2002

(54) Method for massaging human skin

(76) Inventor: Kazuko Uchida, Tkyo (JP)

Correspondence Address: MATTINGLY, STANGER & MALUR, P.C., 1800 DIAGONAL ROAD, SUITE 370, ALEXANDRIA, VA, 22314, US

(21) Appl. No.: 10/070,859

(22) PCT Filed: Jul. 13, 2001
(86) PCT No.: PCT/JP01/06095

(30) Foreign Application Priority Data
Jul. 13, 2000 (JP) 2000-213327

(51) Int. Cl. A61B 19/00

(52) U.S. Cl. 128/898

(57) ABSTRACT

This invention provides a massaging method, wherein, by massaging the facial skin, favorable beauty care and skin care effects, such as promoting the blood circulation and improving the sagginess of the skin, or promoting the firmness of the skin, are obtained. The massage is performed from near the center of the face, in the direction toward the contours of the face, and the facial skin is massaged, starting from below the eyes, passing above the eyes, and subsequently from above the eyes to the cheeks.

### TECHNICAL FIELD

The present invention relates to a method of massaging human faces, which can be used in industrial fields intended for beauty care and skin care. For example, the present invention can be used in the esthetic industry. Furthermore, it can also be used in the beauty care industry, in which the rehabilitation of the skin is achieved by improving the blood circulation, and the firmness and tone of the skin.

### BACKGROUND ART

As a conventional massaging method of this kind, there existed a beauty care method disclosed in the Patent Laid-Open Publication No. HEI 10-113369. FIGS. 1, 10 and 11 which relate to such prior art show the massaging order and the massage lines.

Such prior art provides, for example, a beauty care method, wherein, after massage (a), which is performed in lines extending from near the mouth to the bottom sides of the nose, any of the following massages are performed, namely, massage (b), which is performed on the cheeks, starting from near the mouth, passing the lower lids and drawing circles toward the ears, massage (c), which is performed on the forehead, starting from areas between the eyebrows, passing the upper forehead and drawing arcs toward both ends of the forehead, and massage (d), which is performed on the lower lids, from the inside corners to the outside corners of the eyes; or a beauty care method, wherein, after massage (a) above, massages (b), (c), and (d) are performed in any order. Furthermore, there is an especially favorable facial beauty care method, which suggests performing respectively two or three times, the aforementioned massage (a), which is performed in lines extending from near the mouth to the bottom sides of the nose, the aforementioned massage (b), which is performed on the cheeks, starting from near the mouth, passing the lower lids and drawing circles toward the ears, the aforementioned massage (c), which is performed on the forehead, starting from areas between the eyebrows, passing the upper forehead and drawing arcs toward both ends of the forehead, and then performing two or three times the aforementioned massage (d), which is performed on the lower lids, from the inside corners to the outside corners of the eyes.

According to such conventional method, massages are performed along the blood flow, and various effects are achieved, such as the promotion of blood circulation and improvement in the skin tone, and reduction in the sagginess of the skin.

### DISCLOSURE OF THE INVENTION

When blood circulation is impeded due to fatigue, etc., [the skin] around the eyes, cheeks, etc. sags. This is due to the generation of congested blood in the blood vessels around the eyes and other factors. Therefore, massaging along the blood flow is considered effective as suggested in the aforementioned examples of the prior art. However, after careful study, the inventor of the present invention has found that the massaging method according to the aforementioned examples of the prior art is insufficient, specifically, for achieving on the face the effects of promoting blood circulation or reducing the sagginess of the skin around the eyes.

The purpose of the present invention is to provide a massaging method, in which, by massaging the facial skin, favorable beauty care and skin care effects are achieved, such as the promotion of blood circulation, improvement in the sagginess of the skin, or promotion of the firmness of the skin.

In order to accomplish such purpose, the present invention is characterized in that it is a facial massaging method performed around the eyes, in which the facial skin is massaged, starting from the lower lids, and passing the upper lids, and subsequently from the upper lids to the cheeks.

According to the careful study by the inventor, in order to ensure improvement in the congested blood, or saggy or swollen skin around the eyes, it is effective to massage the blood vessel skin portions around the eyes, along the direction in which the blood vessels extend, in the direction of the blood flow. The massaging method according to the present invention fulfills these conditions.

A preferred embodiment of such massaging method is the massaging method explained above, wherein the massage is performed almost immediately below the lower lids, from near the outside corners of the eyes to near the inside corners of the eyes, and subsequently almost immediately above the upper lids, from near the inside corners of the eyes to near the outside corners of the eyes, and subsequently from the outside corners of the upper lids to the cheeks.

According to an even more preferable embodiment of the present invention, facial skin is massaged, starting from the aforementioned outside corners of the eyes, and descending along the contours of the cheeks. Furthermore, another embodiment of the present invention is a facial massaging method performed around the eyes, wherein a massage is performed along the bottom sides of the nose below the eyes, reaching near the inside corners of the eyes, then passing the forehead near the upper lids, then passing near the outside corners of the eyes or the temples, and reaching the lower lids.

The massages explained above can be performed using a cosmetic agent. Such cosmetic agent is not specifically restricted. As the cosmetic agent, foundations or the cosmetic agents set forth in the aforementioned Patent Laid-Open Publication No. HEI10-113369 can be used.

### BRIEF DESCRIPTION OF THE DRAWINGS

FIG. 1 is a front view of a human face, which shows a specific example of a massage. FIG. 2 is a front view of a human face, which shows a variant of the massage shown in FIG. 1.

### BEST MODE FOR CARRYING OUT THE INVENTION

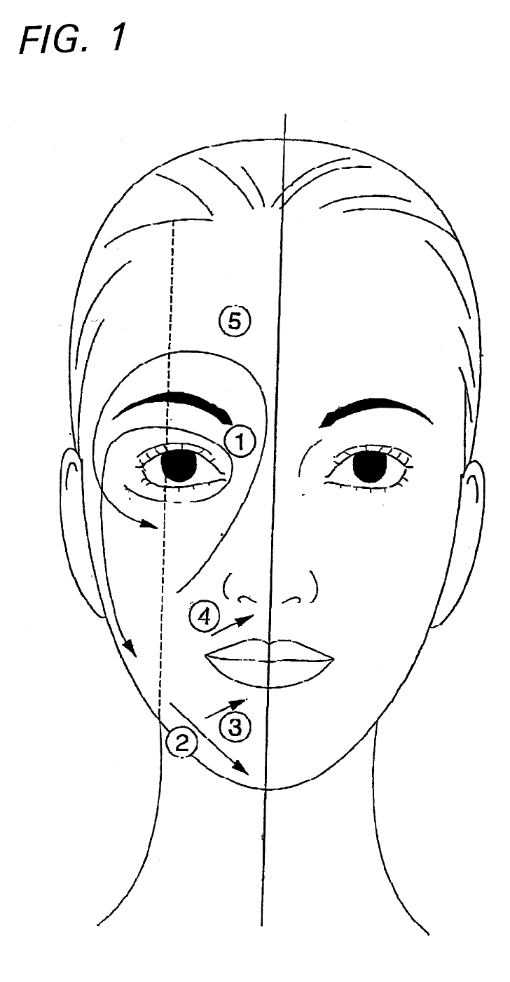

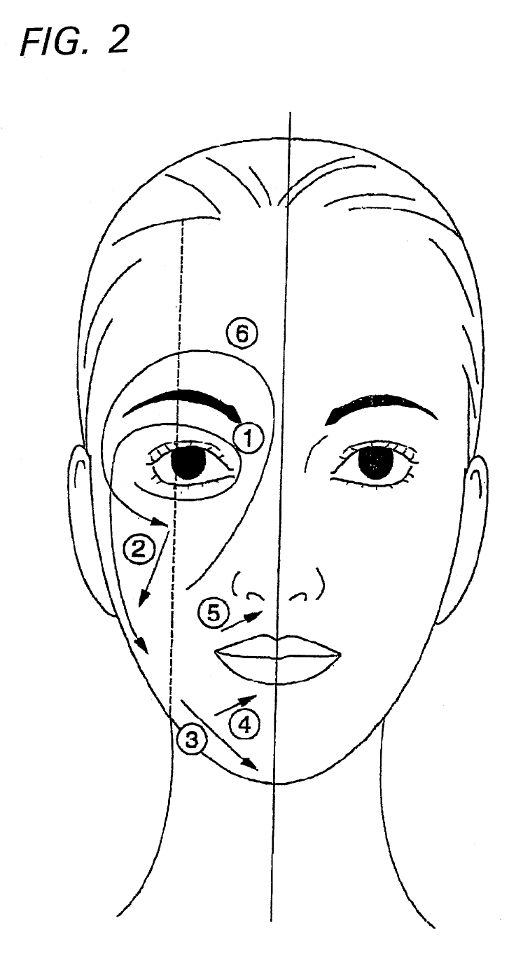

Next, the preferred embodiments of the present invention are explained. FIG. 1 shows the directions and the order of massaging the facial skin. In this Figure, the symbol “→” shows the positions and the directions for performing the massage on the skin, and the numbers show the massaging order.

The first massage line (1) draw arcs and moves in the following manner: the lower lid portions are massaged, starting from the inside corners of the eyes and reaching the outside corners of the eyes; subsequently, the massage line proceeds to the upper lids, and moves along the upper lids, from the inside corners of the eyes to the outside corners of the eyes; and then the massage line descends from the outside corners of the eyes on the upper lids, along the contours of the cheeks, in the direction of the jaw.

Subsequently, the second massage line (2) is a line which is continued from the first massage line, and moves from the lower cheek portions to the lower jaw.

The third massage line (3) is a line which extends from one portion of the second massage line, reaching below the lips.

The fourth massage line (4) is a line which extends from near the upper ends of the lips to the nose.

The fifth massage line (5) starts from near the center portions of the cheeks below the eyes, passes the bottom sides of the nose and then above the eyebrows, and reaches the lower lids, passing the outside corners of the eyes and proceeding toward near the inside corners of the eyes, thereby drawing arcs.

These massage lines follow along the facial blood flow. Specifically, the first massage line has a function of releasing body fluid, such as lymph fluid and blood stagnating around the eyes, into the blood flow (venous flow) in the blood vessels around the eyes, whereby stagnating body fluid, which produces bloated or swollen skin, passes through the blood vessels around the eyes, and is released in the direction of the heart.

While the conventional massaging methods are not performed along the blood vessel lines of the face, especially those around the eyes, the present invention is characterized in that the massage is performed along these blood vessel lines and in the direction of the blood flow.

The massage explained above is applied to the right and the left sides of the face respectively. The massaging order is as shown in FIG. 1, namely, line 1→line 2→line 3→line 4→line 5→. It is desirable for the massage to be performed a number of prescribed times, in such order. The number of times [for performing the massage] is suitably changed according to age or skin conditions. By applying a prescribed cosmetic agent, such as a basic cosmetic agent or foundations, to a cosmetic sponge, putting on make-up during the massage is also possible. The aforementioned order of the massage lines can be suitably changed.

The force which must be applied at the time of the massage can be changed for each massage line. For example, the force applied to thin skin portions, such as around the eyes, can be made smaller than the force applied to other thick skin portions, such as the cheeks. If the aforementioned massage is to be performed, applying a cosmetic agent to a sponge or other cosmetic items, age-related wrinkles, for example, can be made less conspicuous by adjusting the applied force and reducing the amount of cosmetic agent applied to thin skin portions.

### EXAMPLE 1

The testee was a 25-year-old female. After impregnating the sponge with squalane, the massage was performed in the aforementioned order. The massage was repeated a few times in sequence, from line 1 to line 5, on both sides of the face. By comparing the conditions before and after the massage, it was found that the bloatedness of the entire face, such as around the eyes, cheeks, etc., had disappeared.

### EXAMPLE 2

The testee was a 36-year-old female. After performing the massage in the same manner as in Example 1 above, the bloatedness around the eyes had disappeared.

### EXAMPLE 3

The testee was a 45-year-old female. After performing the massage in the same manner as in Example 1 above, the bloatedness around the eyes had disappeared.

FIG. 2 shows Embodiment 2 of the present invention. The difference from Embodiment 1 shown in FIG. 1 is that line (2) is added. After massaging along line (1), a massage is performed along line (2). The massaging order is shown by numbers. Line (2) starts from points below the eyes, which are located on the dotted line running vertically through almost the center of the eye, and extends along the peripheral areas of the cheeks.

By adding this line, the bloatedness at the inside corners of the eyes can be released outside the face, along the blood flow. In the examples above, similar effects were obtained by performing the massage shown in FIG. 2.

### INDUSTRIAL APPLICABILITY

As explained above, by applying the massaging method according to the present invention to human faces, it is possible to provide a massaging method effective for beauty care and skin care, such as promoting blood circulation, improving the sagginess of the skin, or promoting the firmness of the skin. Furthermore, because the massage lines explained above extend along the direction of the facial muscles (facial expression muscles), the massage has the effect of relieving tension in the muscles and tightening the muscles.

1. A facial massaging method performed around the eyes, comprising the steps of:
massaging from near the center of the face, in the direction of the contours of the face, starting from below the eyes and passing above the eyes; and
massaging the facial skin, from above the eyes to the cheeks.

2. A facial massaging method performed around the eyes, wherein a massage is performed on the facial skin, from near the center of the face, in the direction toward the contours of the face, starting from below the eyes, passing above the eyes, and subsequently from below the eyes to the cheeks.

3. A massaging method according to claim 2,
wherein the massage is performed almost immediately below the lower lids, from near the outside corners of the eyes to near the inside corners of the eyes, and subsequently from near the inside corners of the eyes to near the outside corners of the eye, and subsequently from the outside corners of the upper lids to the cheeks.

4. A massaging method according to claim 3,
wherein the massage is performed on the facial skin, starting from said outside corners of the eyes on the upper lids, and descending along the contours of the cheeks.

5. A facial massaging method performed around the eyes,
wherein a massage is performed along the bottom sides of the nose below the eyes, to near the inside corners of the eyes, and subsequently passes the forehead near the upper lids, and further passes near the outside corners of the eyes or the temples and reaches the lower lids.

6. A method according to any one of claims 1 through 5,
wherein a cosmetic agent is applied to a cosmetic item, and said massage is performed, applying said cosmetic agent.
